(19) United States
(12) Plant Patent Application Publication
(10) Pub. No.: US 2023/0309429 P1
(43) Pub. Date: Sep. 28, 2023

(54) Hop Plant Named 'HBC 1134'

(50) ;

(71) Applicant: HOP BREEDING COMPANY, L.L.C., YAKIMA, WA (US)

(72) Inventors: Jason Perrault, Toppenish, WA (US); Michael D. Ferguson, Yakima, WA (US); Scott Varnum, Ellensburg, WA (US); Jaime Avalos P., Granger, WA (US); Scott Herzog, Yakima, WA (US)

(21) Appl. No.: 17/803,908

(22) Filed: Jan. 13, 2023

Related U.S. Application Data
(60) Provisional application No. 63/361,737, filed on Jan. 14, 2022.

(51) Int. Cl. A01H 6/28; A01H 5/02

(52) U.S. Cl. CPC A01H 6/28; A01H 5/02
USPC 800/298

(57) ABSTRACT

A new hop plant named ‘HBC 1134’ is disclosed. ‘HBC 1134’ is used for its exceptional and unique aromatic qualities, and is suitable for beer flavoring.

### STATEMENT REGARDING FEDERALLY SPONSORED RESEARCH OR DEVELOPMENT

None

Genus and species: Humulus lupulus.

Variety denomination: ‘HBC 1134’.

### BACKGROUND OF THE INVENTION

‘HBC 1134’ is a product of a controlled breeding program carried out by the inventors in the Yakima Valley of Washington State. ‘HBC 1134’ was one of several seedlings resulting from a controlled cross made in 2016 with female parent ‘Hersbrucker Pure’, and male parent ‘15.161.49BM’. Seedling plants from this cross were planted in 2017 and screened for disease resistance and sex in a greenhouse and field nursery near Granger, Washington. A single plant of ‘HBC 1134’ was selected in 2017. In 2018 ‘HBC 1134’ was asexually reproduced and expanded to a seven hill plot. In 2020, ‘HBC 1134’ was asexually reproduced via softwood cuttings, and expanded to 30 plants in an evaluation block near Toppenish, Washington. In 2021, ‘HBC 1134’ was further expanded to a one acre test block near Toppenish, Washington with plants asexually reproduced via softwood cuttings. The ‘HBC 1134’ plants have now been observed and evaluated for several years. Throughout several generations of asexual propagation, ‘HBC 1134’ has been observed to retain its distinctive characteristics and remain true to type.

### BRIEF DESCRIPTION OF THE PHOTOGRAPHS

FIG. 1 illustrates a close up of mature leaves and cones of a ‘HBC 1134’ hop plant;

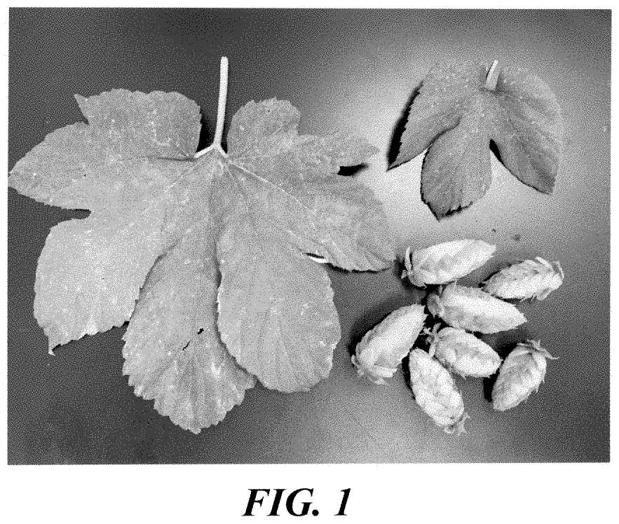

FIG. 2 illustrates a multiple of trellis with maturing ‘HBC 1134’ hop plant bines at approximately three months of growth;

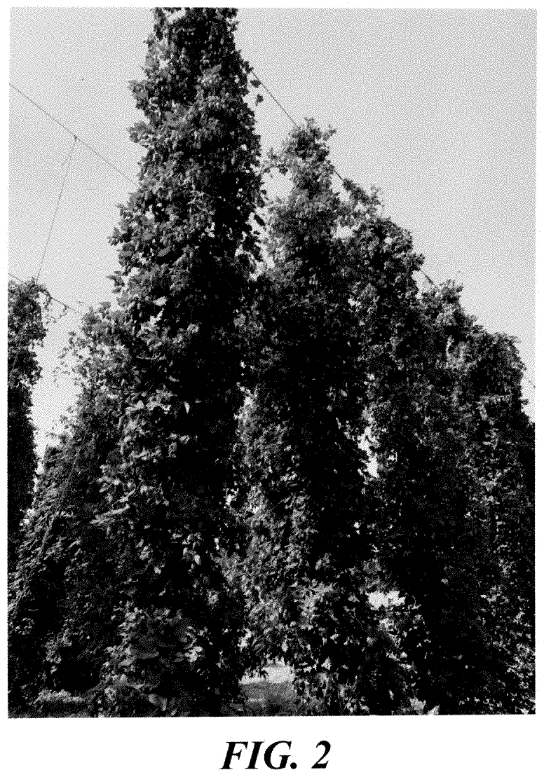

FIG. 3 illustrates a close up of a bine of a maturing ‘HBC 1134’ hop plant at approximately three months of growth on a trellis;

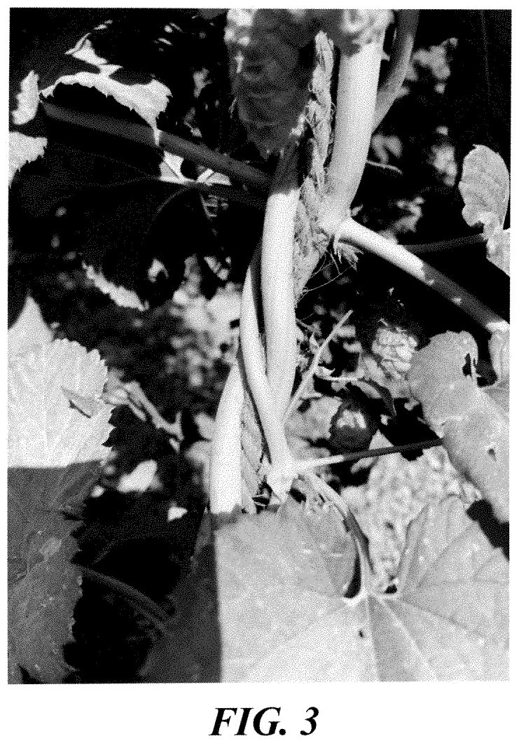

FIG. 4 illustrates cross-sectioned mature cones of a ‘HBC 1134’ hop plant;

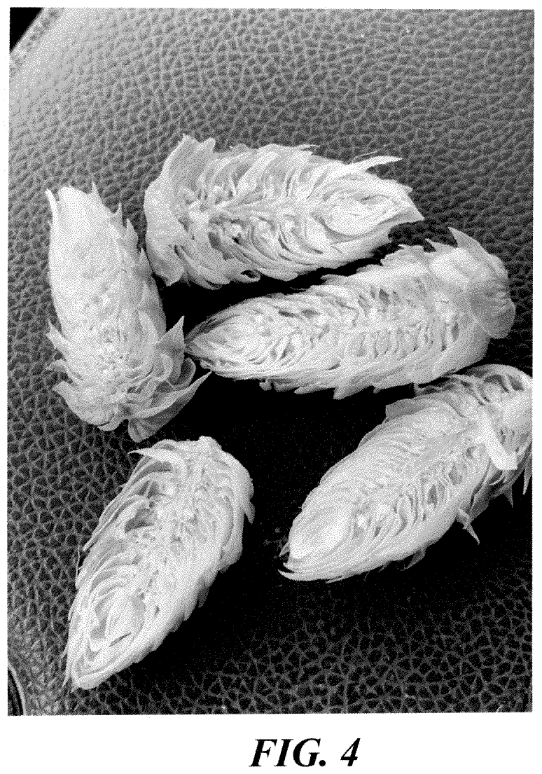

FIG. 5 illustrates a close up of cross-sectioned mature cones of a ‘HBC 1134’ hop plant; and

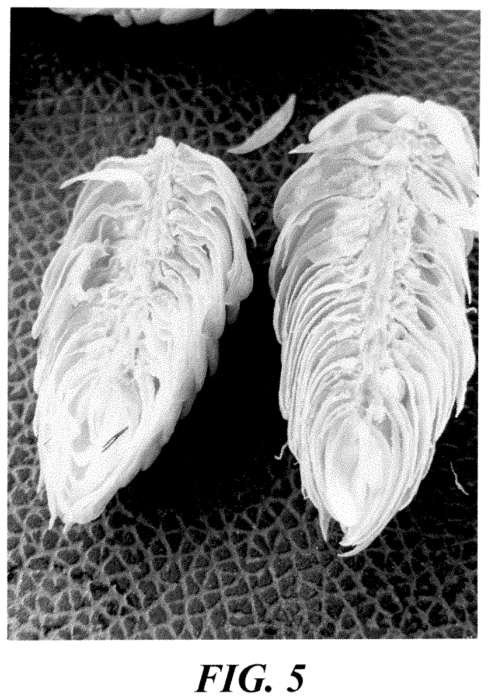

FIG. 6 illustrates a close up of cross-sectioned mature cones of a ‘HBC 1134’ hop plant.

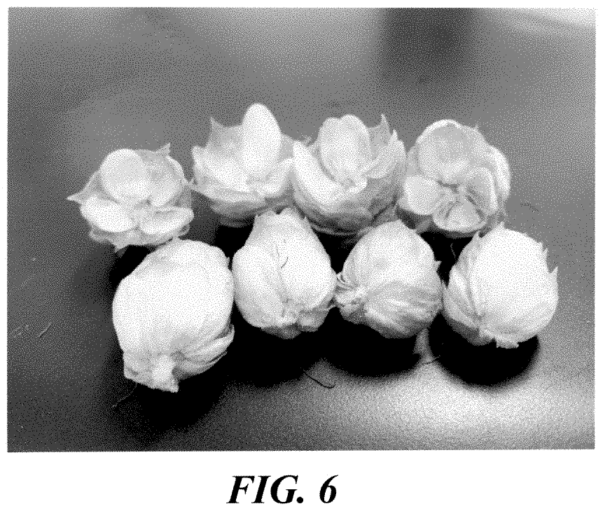

The colors of these illustrations may vary with lighting conditions and, therefore, color characteristics of this new variety should be determined with reference to the observations described herein, rather than from these illustrations alone.

### COMPARISON OF ‘HBC 1134’ TO PARENT PLANT AND COMARISON CULTIVAR

‘HBC 1134’ has many aroma and flavor characteristics to its female parent, the cultivar ‘Hersbrucker Pure’. It is distinguished from ‘Hersbrucker Pure’ by having much higher alpha acids (with alpha acids of approximately 11% percent of cone weight typical for ‘HBC 1134’, versus alpha acids of approximately 5% percent of cone weight typical for ‘Hersbrucker Pure’). Also, ‘HBC 1134’ is much more vigorous, higher yielding, and with a heavier and larger cone size, as compared to ‘Hersbrucker Pure’.

### DETAILED BOTANICAL DESCRIPTION

The following description is based on observations made in February and August of 2021 near Granger, Washington. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. Color code designations are by reference to The R.H.S. Colour Chart, 4th ed., Copyright 2001, published by The Royal Horticultural Society of Great Britain.
Ploidy: Diploid.
Sex: Female.
Use: Brewing beer and ale.
Yield: Average yield is expected to be approx. 2,200 to 3,200 pounds per acre.
Plant shape: Cylindrical.
Growth type: Normal.
Disease and pest resistance or susceptibility:
   Powdery mildew (Podosphaera macularis).—Tolerant.
Oil analytical characteristics (averages):

| | |
|---|---|
| % Alpha Acid (% cone weight) | 10.9 |
| % Beta Acid (% cone weight) | 4.0 |
| % Cohumulone (% alpha acids) | 29.1 |
| Total Oil (ml/100 g of cones): | 2.1 |
| Hop Storage Index (% loss) | 27.1 |
| Aroma Profile | Noble (Spicy, Citrus, Floral, |
| | Woody, and Herbal.) |

Bine:
   Bine color.—Yellow-Green 145C.
   Stripe present.—Yes.
   Stripe color.—Dark Green-Brown 152D.
   Stipule direction.—Interpetiolar.
   Stipule color.—Yellow-Green 145B.
   Stipules per bine.—54.
   Typical bine length.—5.5 m.
   Typical internode length on bine.—12 cm to 15 cm.
   Typical internode length on lateral.—7 cm.
   Bine diameter.—7 mm at base; 7 mm at nine feet; and 7 mm at terminal end of eighteen feet.
   Average lateral length.—65 cm to 75 cm.
   Lateral density at middle third of plant.—Two Laterals per Node.
   Anthocyanin coloration on main bine.—Absent.
Leaf:
   Arrangement.—Opposite.
   Shape.—Palmately Lobed.
   Apex.—Cuspidate.
   Base.—Sagitate.
   Margin.—Serrated.
   Serrations per inch (typically).—5.
   Pose.—Horizontal.
   Average length of mature leaf.—17.5 cm.
   Average width of mature leaf.—15 cm.
   Leaf blistering on the upper side of leaf blade.—Absent.
   Color of mature leaf upper surface.—Green 139A.
   Color of mature leaf lower surface.—Green 139A.
   Color of immature leaf upper surface.—Green 139A.
   Color of immature leaf lower surface.—Green 139A.
   Number of lobes.—7.
   Petiole color at base.—Yellow-Green 139C.
   Average petiole diameter (mature).—3 mm.
   Average petiole length (mature, typical).—4.5 cm.
   Venation.—Palmate.
   Vein color.—Yellow-Green 146C.
Cone:
   Cone shape.—Ovate.
   Average cone length.—3.5 cm.
   Average cone diameter.—1.7 cm.
   Typical cone weight.—0.85 g.
   Bract shape.—Aristate.
   Bract base shape.—Rounded.
   Bract margin.—Entire.
   Bract tip shape.—Cuspidate.
   Bract tip position.—Alternating.
   Degree of bract opening.—Slightly Open.
   Bract tip color.—Green N143C.
   Bract base color.—Green N145C.
   Bract length.—14 mm.
   Bract width.—12 mm.
   Bracteole shape.—Ovate.
   Bracteole length.—9 mm.
   Bracteole width.—14 mm.
   Bracteole color (upper surface).—Green N145B.
   Bracteole color (lower surface).—Green N144C.
   Lupulin glands shape.—Pedunculated Oblong Polyps.
   Lupulin glands color.—Yellow-Orange 21C.
   Number of lupulin glands per cone.—Varies Greatly (Moderate to Many) with approximately 50,000 lupulin glands per Cone.
   Pickability.—Good.
   Time of flowering.—Late July.
   Maturity date range (early, medium, late).—Early.

What is claimed:

1. A new and distinct variety of Hop plant named ‘HBC 1134’, substantially as illustrated and described herein.
